(19) United States
(12) Patent Application Publication
(10) Pub. No.: US 2004/0044295 A1
(43) Pub. Date: Mar. 4, 2004

(54) Graphical user interface for computer-assisted surgery

(75) Inventors: Cynthia Reinert, Montreal (CA); Josiane Crampe, Montreal (CA); Franck Maras, Montreal (CA); Francois Poulin, Montreal (CA)

Correspondence Address: OGILVY RENAULT, 1981 MCGILL COLLEGE AVENUE, SUITE 1600, MONTREAL, QC, H3A2Y3, CA

(73) Assignee: ORTHOsoft INC., Montreal (CA)

(21) Appl. No.: 10/222,832

(22) Filed: Aug. 19, 2002

(51) Int. Cl. A61B 5/103; A61F 2/46

(52) U.S. Cl. 600/587; 434/274; 606/102

(57) ABSTRACT

A computer system and a method for performing a computer assisted surgery uses an expert system driven graphical user interface (GUI) that displays a series of visual display screens that provide information related to respective steps required to perform the surgery. The system displays virtual images of surgical instruments used during the surgery, as well as computer enhanced images of the implant site to assist the surgical team during instrument calibration, implant site planning, implant site preparation, and implant installation.

### CROSS-REFERENCE TO RELATED APPLICATIONS

This is the first application filed for the present invention.

### MICROFICHE APPENDIX

Not Applicable.

### TECHNICAL FIELD

The present invention relates in general to computer-assisted surgery, and, in particular to a system and method for performing a computer-assisted orthopaedic surgical procedure that is guided by an expert system driven graphical user interface.

### BACKGROUND OF THE INVENTION

Orthopaedics is a branch of medicine concerned with diseases, injuries, and conditions of the musculoskeletal system including the bones, muscles, joints, ligaments, tendons, and nerves. A large number of orthopaedic surgeries are performed each day. To be optimally successful and efficient an orthopaedic surgery requires, in addition to a professional surgical team, perfect instruments, imaging support for planning and performing the surgery and precise control of each step of the surgery. These requirements are especially important when performing an orthopaedic surgery using pedicle screws (implants), because a misplaced screw may induce neurovascular damage in a patient. Currently, a screw hole position is assessed by radiologic means and curette palpation. It is recommended that holes be palpated with a curette, or by inserting an electromyographic or fibroscopic probe, before screw insertion. Furthermore, confirmation of screw placement requires intraoperative radiographs. Usually during an orthopaedic surgery more than one screw is placed into a surgical site. The variety of types of orthopaedic surgery requires different surgical instruments and screws in a plurality of sizes and types. All of this makes the job of a surgical team very complicated. Some techniques for surgical operations require a computerized surgical assistance system that employs three-dimensional imaging of the spine and other skeleton articulations in order to simplify the tasks of the surgical team. As is known in the art, insertion of pedicle screws, hip replacements, knee replacements, and various other orthopedic, dental and neurological procedures can be assisted using computer technology.

An example of a computerized surgical assistance system is described U.S. Pat. No. 6,358,245 entitled GRAPHICAL USER INTERFACE FOR ASSOCIATION WITH AN ELECTRODE STRUCTURE DEPLOYED IN CONTACT WITH A TISSUE REGION, which issued to Edwards on Mar. 19, 2002. Edwards describes methods and systems that deploy an electrode structure in contact with the tissue region to perform a gastroenterological surgical procedure. The systems and methods provide an interface, which generates a simplified image of an electrode structure and an indicator image on the simplified image corresponding to a location of the sensor on the electrode structure. The displayed image enables a surgeon to apply energy to heat a tissue region while the images are displayed on the display screen. The displayed image is a virtual image and is used only for schematic illustration of a position of the surgical tool in the patient.

Many other computer-assisted surgery systems are known and widely used, especially systems that are particularly useful or explicitly adapted for use in orthopaedic surgery. While all such systems provide a user interface, they depend on the expertise of the surgeon to guide the surgical process. As is well known, modern surgery is performed by skilled teams that cooperate to accomplish the task as quickly and efficiently as possible. However, current computer-assisted surgery systems lack an expert system core that is adapted to capitalize on the expertise of team members.

There therefore exists a need for a computer-assisted surgery system with a graphical user interface that can be used in an operating room to guide and assist a surgical team during a surgical procedure.

### SUMMARY OF THE INVENTION

It is therefore an object of the invention to provide a computer-assisted surgery system with a graphical user interface (GUI) adapted to guide a surgical team through a surgical procedure.

In accordance with an aspect of the invention, there is provided a graphical user interface (GUI) for guiding a surgical team performing a computer-assisted surgical procedure. The GUI includes a series of visual display screens for providing information related to respective steps required to perform the surgical procedure, and for displaying to the surgical team representations of selected surgical instruments used during the surgical procedure. The GUI also provides means for permitting the surgical team to advance through the series of visual display screens as each of the respective steps is completed.

In accordance with another aspect of the invention there is provided a method of guiding a surgical team in the performance of a computer-assisted surgical procedure using a GUI. The method comprises a first step of providing the surgical team with information related to respective steps required to perform the surgical procedure using the GUI. In a second step, representations of selected surgical instruments used during the surgical procedure are displayed. Virtual images, of a part of a patient that is subject to the surgical procedure, are also displayed. The GUI permits the surgical team to advance through a series of visual displays engineered to guide the surgical procedure, as each respective step of the surgical procedure is completed.

In accordance with yet another aspect of the invention there is provided a computer-assisted surgical system, the system comprises a computer including a display monitor having a graphical user interface (GUI) for guiding a surgical team performing the computer-assisted surgical procedure. The GUI includes a series of visual display screens for providing information related to respective steps required to perform the surgical procedure, and for displaying to the surgical team virtual images of selected surgical instruments used during the surgical procedure in relative alignment with images of part of a patient subject to the surgical procedure. The system further comprises means for determining a location of the selected surgical instruments with respect to the part of the patient, and means for acquiring images of the part of the patient and processing the images to generate the virtual three-dimensional images of the part of the patient. The system further comprises means for permitting the surgical team to advance through the series of visual display screens as each respective step of the surgical procedure is completed.

### BRIEF DESCRIPTION OF THE DRAWINGS

Further features and advantages of the present invention will become apparent from the following detailed description, taken in combination with the appended drawings, in which:

FIG. 1 schematically illustrates a system for performing computer-assisted surgery (CAS) that includes a graphical user interface (GUI) in accordance with the invention;

FIG. 2 is a flow chart of principal steps of a method for guiding the surgical team in performing a CAS procedure using a GUI;

FIGS. 3A and 3B schematically illustrate an organization of principal display screens of the GUI component of the system shown in FIG. 1;

FIG. 4 is a schematic illustration of a sequence of display screens displayed by the GUI for calibrating surgical instruments in preparation for a surgical procedure;

FIG. 5 is a schematic illustration of a sequence of display screens used to validate images of a part of a patient that is subject to the surgical procedure;

FIG. 6 is a schematic view of a display screen displayed by the GUI for guiding a surgical team during the planning of an implant site; and

FIG. 7 is a schematic view of a display screen displayed by the GUI for guiding the surgical team through a procedure for insertion of an implant.

It should be noted that throughout the appended drawings, like features are identified by like reference numerals.

### DETAILED DESCRIPTION OF THE PREFERRED EMBODIMENT

The invention provides a method and system that uses an expert system driven graphical user interface (GUI) for guiding a surgical team through a computer-assisted surgery (CAS) procedure.

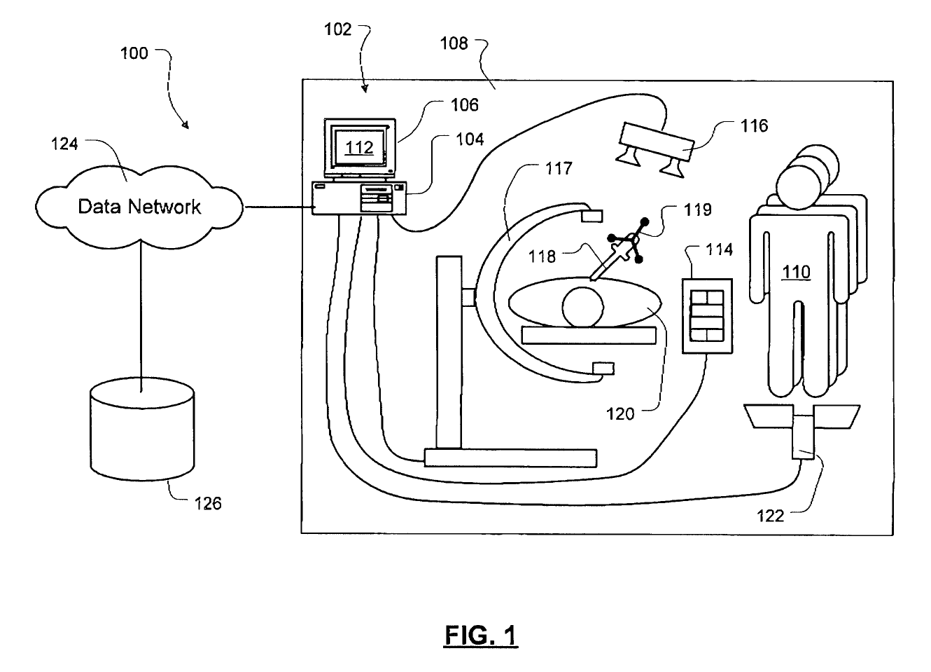

FIG. 1 shows an exemplary embodiment of a system 100 for performing a CAS, in accordance with the invention. The system 100 includes a computer 102 in an operating room 108. The computer 102 has a processor 104 for executing a CAS application and a display monitor 106. The display monitor 106 displays information to a surgical team 110 using a graphical user interface (GUI) 112 that presents a series of visual display screens associated with respective steps required to perform the surgical procedure. The visual display screens display virtual images of selected surgical instruments 118 used during the surgical procedure and three-dimensional images of a part of a patient 120 that is subject to the surgical procedure. A manual input device 114 is preferably connected to the computer 102 to permit the surgical team 110 to input commands to the CAS program for advancing through the series of visual display screens of GUI 112, as each of the respective steps of the surgical procedure is successively completed. The manual input device 114 is preferably adapted to be easily sterilized and is resistant to fluid contamination. A similarly adapted foot-operated input device 122 may also be connected to the computer 102. The surgical team 110 may choose to operate either the manual input device 114 or the foot-operated device 12, as required. The computer 102 is connected to an imaging system that includes a binocular video camera 116 for locating and tracking movement and orientation of the surgical instrument 118, as will be explained below in more detail.

An X-ray imaging system, such as a fluoroscope 117, acquires the images of a part of a patient 120 that is subject to the surgical procedure. As many images as are sufficient for the task at hand can be taken in accordance with the present invention. The images are processed by the CAS to provide virtual three-dimensional images used to guide the surgical team 110 through the surgical procedure, as will also be explained below in more detail. The computer 102 may also be connected to a data network which may be a data network such as the Internet 124 or a local area network (LAN) for accessing expert systems or applications 126 stored externally to the operating room 108.

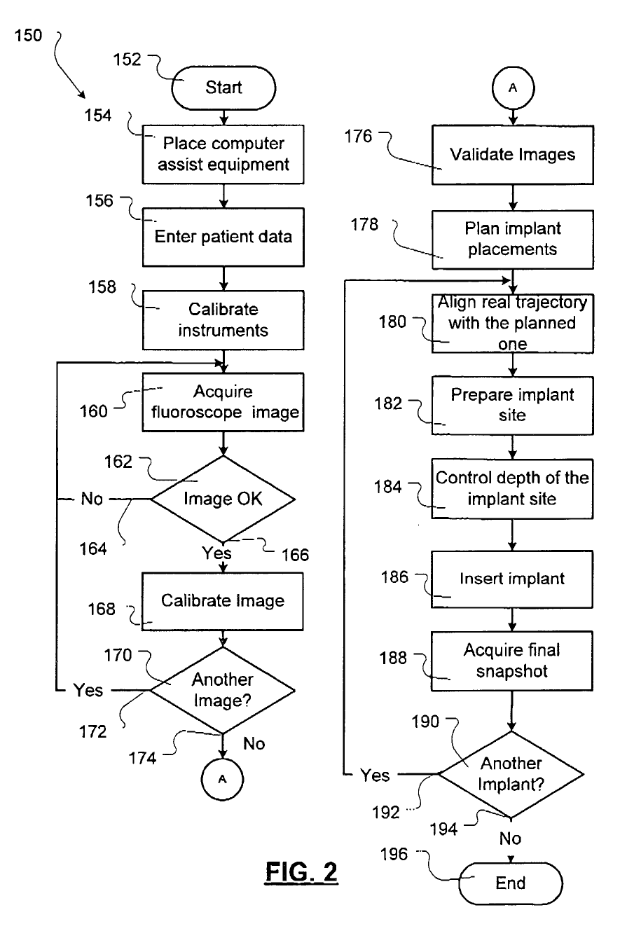

The invention also provides a method for guiding the surgical team 110 in performing a CAS procedure using the GUI 112. A general overview of the method is described with reference to a flow chart 150 shown in FIG. 2. The method provides the surgical team 110 with information related to respective steps required to perform the surgical procedure using the GUI 112. The surgical team 110 can view virtual images of selected surgical instruments used during the surgical procedure and images of a part of a patient 120 that is subject to the surgical procedure. Further, a series of display screens displayed by the GUI 112 guide the surgical team 110 through the CAS procedure, as each of the respective steps is completed. One embodiment of the GUI 112 provides visual and audio information related to instrument calibration, patient imaging, implant site planning, implant site preparation, and implant installation.

The method starts (step 152) by placing the CAS equipment 102 in the operating room 106 (step 154). Patient data, surgery type and other information is then entered into the computer 104 (step 156). The surgical instruments 118 are then calibrated (step 158). During the calibration process, the surgical team 110 is prompted to identify a surgical instrument to be calibrated and to connect a three-dimensional instrument locator 119 (FIG. 1) to the identified instrument 118. When the identified instrument 118 with the connected locator 119 is moved into a field of view of a camera 116 used to acquire images of the instrument locator, the CAS application calibrates the instrument. The surgical team 110 is then prompted and guided to acquire (step 160) at least two fluoroscopic images of the part of the patient subject to the surgical procedure. The images are then verified (step 162).

Menu options are provided to permit the surgical team 110 to transform images by selecting options presented by the GUI 112 to rotate, flip or restore an image. If an image is not satisfactory, the GUI 112 returns 164 to step 160 to permit new images to be acquired. Otherwise, the GUI 112 continues 166 to step 168. Next, the image is calibrated (step 168). If more images are required for the surgical procedure (as determined in step 170) then the GUI 112 returns 172 to step 160, otherwise the GUI 112 continues at 174 to step 176. The images are validated (step 176) as the surgical team 110 is prompted and guided through a procedure for validating the respective images. The GUI 112 prompts the surgeon to place one of the calibrated instruments on the part of the patient that is subject to the surgical procedure, and to compare an actual location of the surgical instrument 118 with a virtual representation of the instrument relative to an image of the part of the patient displayed by the GUI 112. The surgical team 110 is then guided through the planning of implant placements (step 178). The GUI 112 prompts the surgical team 110 to place a calibrated instrument on the part of the patient where an implant is to be inserted, select a type and size of implant to be inserted, and displays, in at least two views of the part of the patient, a virtual image of the selected implant. The position and orientation of the virtual image of the instrument and the implant, is aligned with respect to the images of the patient, to permit the surgical team to evaluate the planned implant site and selected implant type and size.

To prepare the implant site, a calibrated instrument (a drill guide, for example) is positioned in alignment with the planned implant trajectory (step 180). The GUI 112 guides the surgeon through the preparation of the implant site (step 182) by displaying menu options to permit the surgical team 110 to select an implant site preparation option, and displays, in at least two views of the part of the patient, a virtual image of the selected instrument and its alignment with a planned location of the implant, to permit the surgical team 110 to prepare the implant site so that the implant can be inserted into the planned implant site. During the preparation of the implant site 182, the GUI 112 displays a visual guide used to indicate to the surgical team 110 a distance of travel of the instrument used to prepare the implant site. The GUI 112 dynamically updates the visual display to indicate to the surgical team 110 the distance of travel of the instrument as the instrument is used to prepare the implant site (step 184). The GUI 112 then prompts the surgical team 110 to insert the implant (step 186). The GUI 112 displays a menu option to permit the surgical team 110 to select an implant insertion option, select the instrument required to insert the selected implant, and displays, in at least two views of the part of the patient, a virtual image of the selected implant as it is inserted. The virtual path of the implant is computed by the CAS application by tracking a path of the instrument used to insert the implant into a prepared implant site. After the implant is inserted, a snapshot is acquired (step 188), in accordance with the present embodiment. Preferably snap shots may also be taken at other times during the procedure using a predefined command. If another implant is to be inserted (step 190), then the method returns at 192 to step 180. Otherwise, the GUI 112 presents a menu option to end the CAS application (step 196) after menu options are displayed to permit the surgical team 110 to manipulate the GUI 112 to clear an images bank after the surgical procedure is completed, to permit the image memory to be used for other surgical procedures.

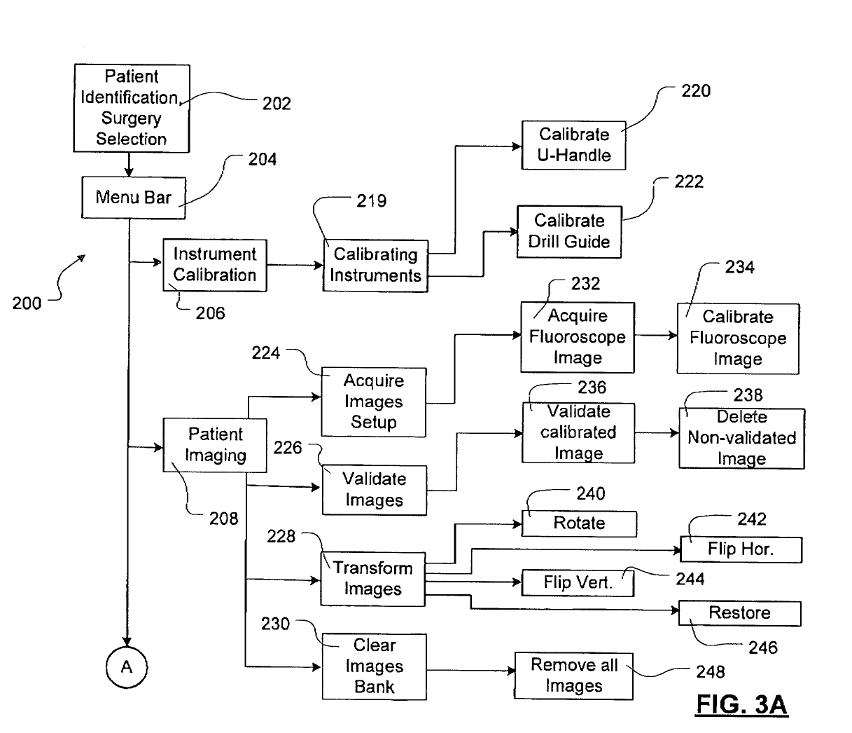

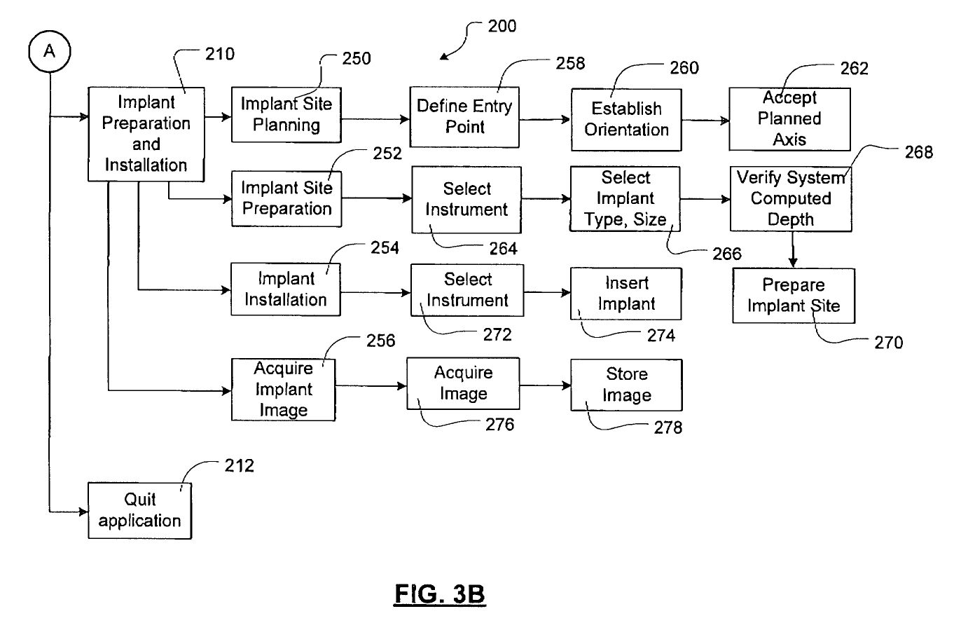

FIGS. 3A, 3B illustrate principal display screens of a menu bar 204 of the GUI 112 in accordance with the present invention. As shown in FIGS. 3A, 3B, the GUI 112 may be implemented as a multi-layer menu-tree providing the surgical team 110 with menu-driven access to the functionality of the CAS application.

In the embodiment illustrated in FIGS. 3A, 3B, the menu-tree enables the surgical team 110 to access three general categories of functionality, namely an “Instrument Calibration” selection 206 for accessing functionality related to a calibration of selected instruments to be used during the surgical procedure; a “patient Imaging” selection 208 for accessing functionality related to acquiring, processing and validating images; an “Implant Preparation and Installation” selection 210 for accessing functionality related to implant site planning, implant site preparation and implant installation. A “Quit Application” selection 212 is also provided to permit the surgical team 110 to cancel or terminate the CAS application. Other selections may also be provided in the menu-tree to provide access to other desired functions of the CAS application such as, for example, context-sensitive help, etc. Within each category of functionality, the menu-tree 200 may be populated as required to enable rapid and intuitive access to the functionality of the CAS application.

As shown in FIGS. 3A and 3B, the surgical team 110 must enter “Patient Identification and Surgery Selection” 202 information before a GUI 112 is selected by the CAS. As its name implies, a patient to be operated on is identified, the type of operation is specified and an identification of each member of the surgical team is recorded, along with any other documentary information required. The type of operation is used by the CAS to select an expert system driven GUI 112 to be displayed to the surgical team 110. Consequently, the type of operation determines a configuration of the remainder of the GUI 112, which may differ from the exemplary structure described with reference to FIGS. 3A and 3B.

The “Instrument Calibration” selection 206 of the illustrated embodiment presents a “Calibrate Instruments” menu 219 that, in this exemplary embodiment, includes two options, namely “Calibrate U-Handle (Universal Tool Handle)” 220 and “Calibrate Drill Guide” 222, which are required for orthopaedic spinal surgery. As will be understood by those skilled in the art, more or different instruments may be required for other surgeries such as hip or knee replacements, for example.

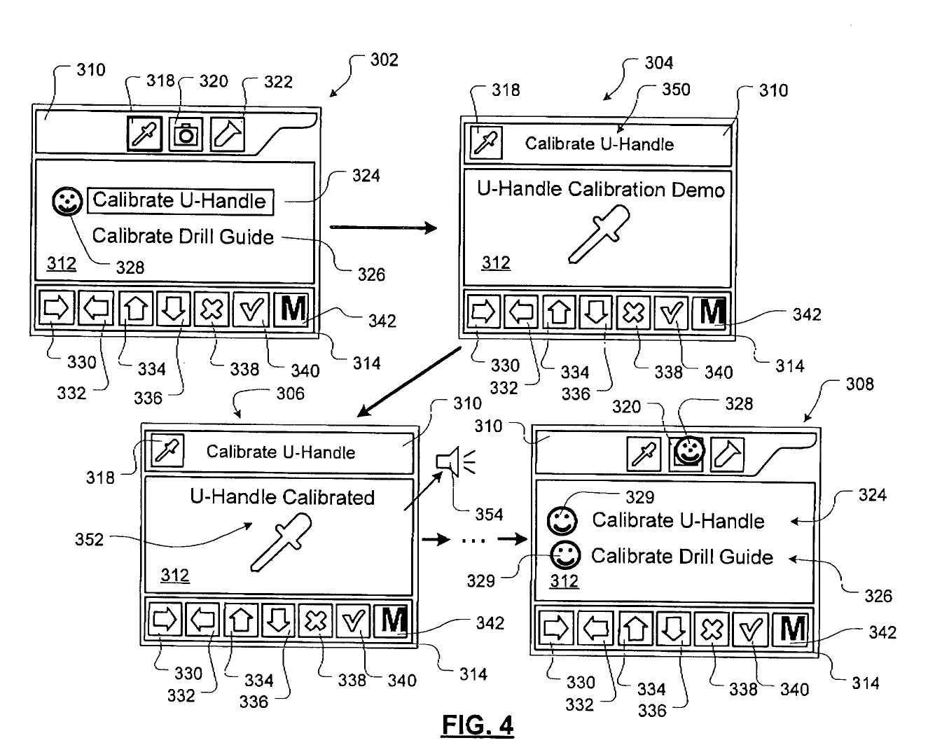

As mentioned above, the “Patient Imaging” selection 208 of the menu bar 202 is used to access functionality of the CAS related to the capturing and processing of images of a surgery site, as will be explained below in more detail with reference to FIG. 4. The “Patient Imaging” selection 208 includes an “Acquire Images Setup” menu option 224, a “Validate Images” menu option 226, a “Transform Images” menu option 228 and a “Clear Images Bank” menu option 230.

The “Acquire Images Setup” menu option 224 is used to control an imaging system of the CAS system ready to acquire images of the surgery site. When the imaging system is ready an “Acquire Fluoroscope Image” option 232 enables the surgical team to acquire fluoroscope images of the surgery site and a subsequent “Calibrate Fluoroscope Image” option 234 enables the surgical team to calibrate the acquired images. The calibrated images are used by the CAS application to generate an image of the surgery site.

The “Validate Images” option 226 enables the surgical team to validate a generated image by comparing virtual points on the image with real points on the surgery site. The surgical team, using the “Validate Images” option 226, can validate the generated three-dimensional image using a “Validate calibrated Image” option 236. After a surgeon of the surgical team has compared a generated image with the real points on the surgery site, the surgeon can accept the generated image or discard it. An accepted image is automatically saved in an image bank reserved for the surgery. A discarded image can be deleted from the CAS application using a “Delete Non-Validated Image” option 238.

After the generated images are validated, the “Transform Images” option 228 permits the surgical team to modify images by selecting options to rotate an image 240, flip it horizontally 242 or flip it vertically 244, or restore a transformed image 246.

The “clear Images Bank” option 230 enables the surgical team to delete all images acquired for the surgical procedure. The GUI 112 preferably displays the images in the images bank and permits the surgical team 110 to delete select images. A “Remove all Images” option 248 permits the surgical team to delete all images from the images bank. The Remove all Images option 248 is generally used to restart image acquisition because of an inadvertent dislocation of a position reference tool affixed to the patient, or when the application is restarted. Further options may be required to provide for the resetting of a tracking system that provides a trace of an instrument, etc.

The “Implant Preparation and Installation” menu 210 provides an “Implant Site Planning” option 250 for accessing functionality for defining an entry point for an implant insertion and an axis of orientation of the implant. An “Implant Site Preparation” option 252 permits the surgical team 110 to prepare the implant site to receive the implant. An “Implant Installation” option 254 permits the surgical team to insert the implant into the prepared implant site. An “Acquire Implant Image” option 256 permits the surgical team 110 to acquire one or more images of the inserted implant.

As noted above, using the “Implant Site Planning” option 250, the surgical team 110 can plan the implant site by first defining an entry point for the implant using a “Define Entry Point” option 258. Using the “Establish Orientation” option 260 the surgeon can generate a virtual trajectory of an axis of the implant site. After the axis is defined, the surgeon can use an “Accept Planned Axis” option 262 to save the planned parameters of the implant site.

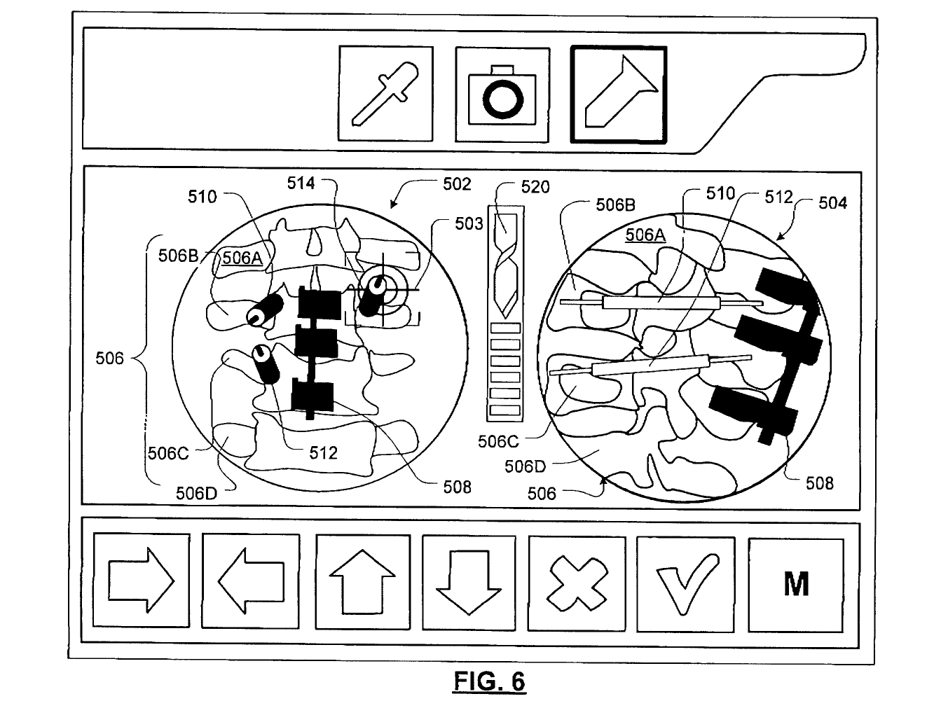

The “Implant Site Preparation” menu selection 252 guides the surgical team through the preparation of the implant site. A “Select Instruments” option 264 permits the surgical team to select a surgical instrument for preparing the implant site. A “Select Implant Type, Size” option 266 is selected to define specific characteristics of the implant, including its type and size. After the “Select Instrument” option 264 and “Select Implant Type, Size” option 266 are successfully completed, a “Verify System Computed Depth” option 268 is presented to the surgeon. The CAS 100 computes a depth of the implant site using the selected implant data and displays the computed depth on a depth chart, as will be explained below with reference to FIG. 6. The surgeon can either accept the computed depth, or change it by changing the selected implant, or by adjusting the depth to accommodate a special circumstance. A “Prepare Implant Site” option 270 enables the surgeon to prepare the implant site by, for example, drilling a bore having a longitudinal axis, and depth that matches the planned implant site, as will be explained below with reference to FIG. 6.

The “Implant Installation” menu 254 guides the surgeon through the process of inserting the implant into the prepared implant site. A “Select Instrument” option 272 permits the surgeon to select an instrument, a screwdriver, for example, for inserting the implant into the prepared implant site. An “Insert Implant” option 274 is used to guide the surgeon through a process of inserting the implant into the prepared implant site. The GUI 112 guides the surgeon through the process of inserting the implant in real time by displaying three-dimensional images of the surgery site, over which virtual images of the prepared implant site are displayed. As the implant is inserted using the selected tool, a virtual image of an actual path of the inserted implant is displayed over the virtual image of the prepared site.

After the insertion of the implant(s) is completed, surgical records may be completed by acquiring images of the implant(s). An “Acquire Implant Image” selection 256 for acquiring an image of the installed implant includes an “Acquire Image” option 276, which permits the surgical team 110 to use the imaging unit 117 to capture an image of the surgery site with the installed implant. Preferably the surgical team 110 is prompted to takes images as useful for recording the procedure. The same command can be used at other junctures to acquire images as desired by the surgeon, for documentation or other reasons. A “Store Image” option 278 prompts the CAS application to save the image of the installed implant. A “Quit application” selection 212 permits the surgical team to quit CAS application.

Having described a general structure of the menu-tree shown in FIGS. 3A, 3B, an embodiment of selected portions of one implementation of the GUI 112 is further described below with reference to FIGS. 4-7, in order to illustrate in general terms how the expert system driven GUI 112 can guide a surgical team 110 through a surgical procedure.

FIG. 4 illustrates an exemplary series of display screens displayed by GUI 112 on the computer monitor 106 to guide a surgical team 110 through the instrument calibration process. The display screens shown in FIG. 4 are based on the structure of the menu-tree shown in FIGS. 3A and 3B.

As seen in FIG. 4, in one embodiment of the GUI 112 in accordance with the invention, the display screens (302, for example) are organized so that a top portion of the respective screens displays a menu bar 310. At a top level, the menu bar 310 displays icons 318-322 representative of the three menu selections shown in FIGS. 3A, 3B, namely Instrument Calibration 206 (icon 318), Patient Imaging 206 (icon 320), and Implant Preparation and Installation 210 (icon 322). A highlighted border around a selected icon (see 318) indicates that the menu selection is selected and the selection is accepted by activating the accept key 340. The icon selection prompts the GUI 112 to display a next level display screen 304, which is a first display screen in the Calibrate Instrument sub-tree. This organization is consistent throughout this embodiment of the GUI 112, but is not intended as a limitation on the invention. As will be obvious to those skilled in the art, any number of layouts that provide the above named functionality in a user friendly and accessible manner can equally be used in embodiments of the invention.

After patient identification and surgery selection 202 (FIG. 3A), and selection of the “Instrument Calibration” icon 318, the GUI 112 displays to the surgical team 110 a sequential sub-tree of visual display screens 302, 304, 306 and 308, for guiding the surgical team 110 through the instrument calibration process.

Each visual display screen of the GUI 112 includes a menu bar 310, an information pane 312 and a command bar 314. The information pane 312 sequentially displays actions that are selected by an expert system in dependence on the type of surgery that is to be performed. In the illustrated example the information pane 312 of the visual display screen 302 includes a “Calibrate U-Handle” 324, a “Calibrate Drill Guide” 326 and a to-do smiley icon 328.

The command bar 314 includes a number of icons. A “Back” icon 330, a “Forward” icon 332, an “Up” icon 334 and a “Down” icon 336 permit the surgical team 110 to manoeuvre forwards and backwards through the GUI 112, or up and down through a respective display screen. A “Cancel” icon 338 and an “Accept” icon 340 permit the surgical team 110 to cancel or accept a specific selection, option or action. Preferably icons such as the Accept Icon 340 can be displayed in three states: active, disabled or recommended. An active state is displayed when available; a disabled state indicates that the Accept Icon 340 cannot be selected; and recommended indicates that given a current state, the button is expected to be used next. A “Main menu” icon 342 returns the surgical team 110 to a main application menu, (not shown) that is organized in menu pages.

Upon selecting the “Instrument Calibration” icon 318, the icon is highlighted and the information pane 312 displays a “Calibrate U-Handle” 324, a “Calibrate Drill Guide” 326 and the to-do smiley icon 328. The to-do smiley icon 328 indicates a next action to be performed, in this case the calibration of the U-handle. One of the menu selections “Calibrate U-Handle” 324 and “Calibrate Drill Guide” 326 can be selected by clicking either of icons 330 or 332. After selecting the “Calibrate U-Handle” 324 and clicking the “Accept” icon 340, the information pane 312 of the display screen 304 is displayed. The menu bar 310 of the display screen 304 displays the icon 318 and text “Calibrate U-Handle” 350. The information pane 312 displays a virtual image of a U-Handle and plays an animated demonstration (not shown) of the steps that must be performed by the surgical team 110 to calibrate the U-Handle. The demonstration permits team members to calibrate the instrument while the surgeon performs other pre-operative or operative tasks. By following the steps displayed by the GUI 112, the surgical team presents the U-Handle with attached locator 119 (FIG. 1) to the view of the binocular camera 116, which detects radiation from an infrared source (not shown) reflected by the locator 119, and the CAS application records the identity of the U-Handle, which is associated with a geometry of the locator 119 in a manner well known in the art. Of course other methods for calibration can equally be used in accordance with other embodiments of the invention, including, but not limited to, the use of electromagnetic sensors. After the U-Handle has been calibrated, the display screen 306 is displayed and an audio tone 354 is generated to inform the surgical team 110 that the U-Handle has been successfully calibrated. The information pane 312 of display screen 306 displays the text “U-Handle Calibrated” along with a virtual image of the calibrated tool 352.

The surgical team 110 can then progress to a next step by selecting the “menu” button 314 from the command bar 314 to indicate that the team is ready to move to a next step in the procedure. The same steps that were followed to calibrate the U-Handle are followed to calibrate a drill guide. The task-completed smiley icons 329 that are displayed in the information pane 312 of the display screen 308 indicate that both the U-Handle and the drill guide have been successfully calibrated. The to-do smiley 329 displayed in the menu bar 310 of display screen 308 indicates that the next stage in the surgical procedure is the patient imaging stage.

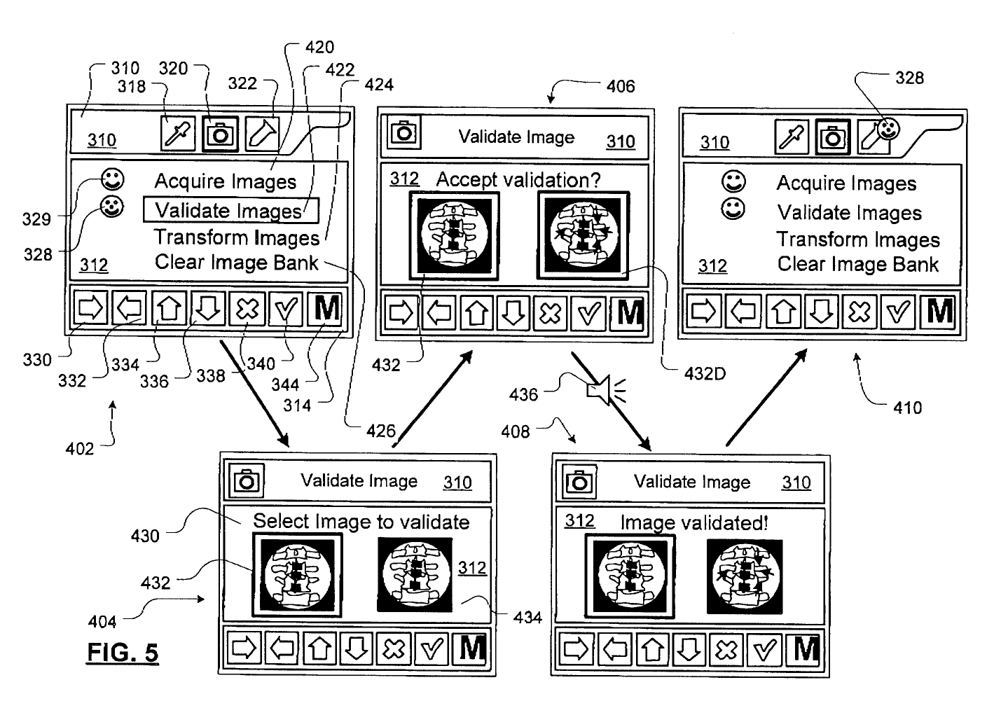

FIG. 5 shows an exemplary series of display screens displayed by GUI 112 to guide the surgical team 110 through steps required to validate images of the part of the patient that is subject to the surgery. The screens displayed by the GUI during an image acquisition step in the procedure are not shown.

The information pane 312 of the display screen 402 includes an “Acquire Images” option 420, a “Validate Images” option 422, a “Transform Images” option 424, a “Clear Image Bank” option 426, and two smiley icons 328, 329. The task-completed smiley icon 329 indicates that the images have been acquired. The to-do smiley icon 328 indicates that the validate images function is the next task to be performed.

After selecting the “Patient Imaging” icon 320, the GUI 112 displays a sequential sub-series of display screens. The “Patient Imaging” menu displayed in the information pane 312 of display screen 402 presents all of the patient imaging options described above. If the validate images option is selected, the information pane 312 of display screen 404 prompts the surgical team 110 to select an image to validate. After the image is selected, display screen 406 prompts the surgeon to indicate whether the selected image has been validated, as will be explained below in more detail. If the surgeon indicates that the image has been validated, an auditory tone 436 is played and, display screen 408 confirms the image validation before the image is stored in the image bank.

On selecting the “Validate Images” option 422, the GUI 112 displays the display screen 404 having an information pane 312 that displays up to two acquired images at a time, in accordance with the present embodiment. The surgical team 110 selects an image to be validated by pressing the left arrow 330 or the right arrow 332 to select one of the two images, which correspond to orthogonally oriented pictures in accordance with the present invention. The surgical team 110 then scrolls through the selected image using the up and down arrows, and presses the accept icon 340 to continue to the next display screen 406 when the desired view is displayed. The surgical team 110 is presented with an acquired image 432 and a demonstration image 432D. The demonstration image 432D displays arrows indicating points suggested by the expert system to be used to validate the image 432. The surgeon places a calibrated instrument 118 (FIG. 1) on corresponding points on the patient. As the surgeon places the calibrated instrument (the Universal-Handle (U-Handle) with an awl tip, for example), the CAS computes a position of the awl tip with respect to the validation image 432 and the GUI 112 displays a virtual image of the instrument on the validation image 432. The surgeon then compares an actual location of the calibrated instrument with the virtual representation of the instrument on the validation image 432. If the actual location of the calibrated instrument 118 at the plurality of points on the part of the patient is substantially the same as the position of the virtual representation of the instrument on the validation image 432, the image is valid and can be used for surgical purposes. The surgical team 110 accepts the validated image by pressing the accept icon 340. The audio tone 436 is sounded to indicate that the validated image has been saved. This procedure is repeated until all images required for surgery have been validated. After all images are validated and the menu button 344 is selected, the patient imaging menu is re-displayed with a task-completed smiley icon 329 beside the validate images option, and a to-do icon 328 is displayed over the implant menu selection 322, to indicate the next step in the surgical procedure.

FIG. 6 illustrates an example of a display screen with an information pane 312 that is displayed by GUI 112 during an implant planning stage of the surgical procedure. The information pane 312 displays a validated anterior position (AP) image 502 and a lateral (LAT) image 504 of the part of the patient that is the subject of the surgery. In this example, a plurality of vertebrae 506 includes a first vertebra 506A, a second vertebra 506B, a third vertebra 506C and a fourth vertebra 506D. The first, second and third vertebrae support clamps 508, which in turn support a locator 119 (not shown) of a unique geometry used by the CAS application to track a relative position of the part of the patient subject to the surgery.

The GUI 112 displays this information pane when a menu option for implant site planning 250 (FIG. 3B) is selected. Implant site planning permits the surgeon to select implant points by placing a calibrated instrument, such as the U-Handle with an awl tip on selected vertebra when the information pane shown in FIG. 6 is displayed by the GUI 112. As shown in FIG. 6, planned implant sites 510 and 512 have already been selected In order to plan an implant site, the instrument is placed on the vertebra and oriented until an entry point shown in the AP image 502 and an axis of orientation shown in the lateral image 504 are oriented to the surgeon's satisfaction. Although the two-dimensional imagery required for the drawing shown in FIG. 6 cannot adequately display the visual effect of a three-dimensional image, implant site planning permits the surgeon to locate the entry point and orientation of respective implant sites with precision.

When an implant site is in the planning stage, a cursor 503 indicates the entry point in the AP view 502, while an axis of orientation of the implant is shown in the lateral view 504, as explained above. After an entry point and an acceptable axis of orientation have been established by the surgeon, the surgeon confirms the implant placement by selecting the “Accept” button 340. When the accept button is selected, the CAS computes a hole depth for the implant to be inserted using implant selection information input by the surgeon. The depth of the hole to be drilled to receive the implant is graphically displayed on a depth gauge 520. The surgeon may accept the computed depth or adjust it as required. If accepted, the surgeon selects the “Accept” button 344 and the planning for the implant site is completed. This pre-planning of implant sites permits the surgeon to choose an optimal location for each implant, thus improving the probability of a successful operation.

After each of the required implant sites have been planned, pedicle holes are drilled using for example an electrical drill known in the art, with the direction of the drill guide calibrated as described above with reference to FIG. 4. As the pedicle hole is bored, a virtual path of the drill bit is displayed in a contrasting colour over the planned implant site 510, for example, and concurrently, a depth of the pedicle hole is shown on the depth gauge 520 as the hole progresses. This permits the surgeon to monitor an axis of orientation and a depth of the hole concurrently to ensure that the pedicle hole is accurately placed and drilled to an exact required depth.

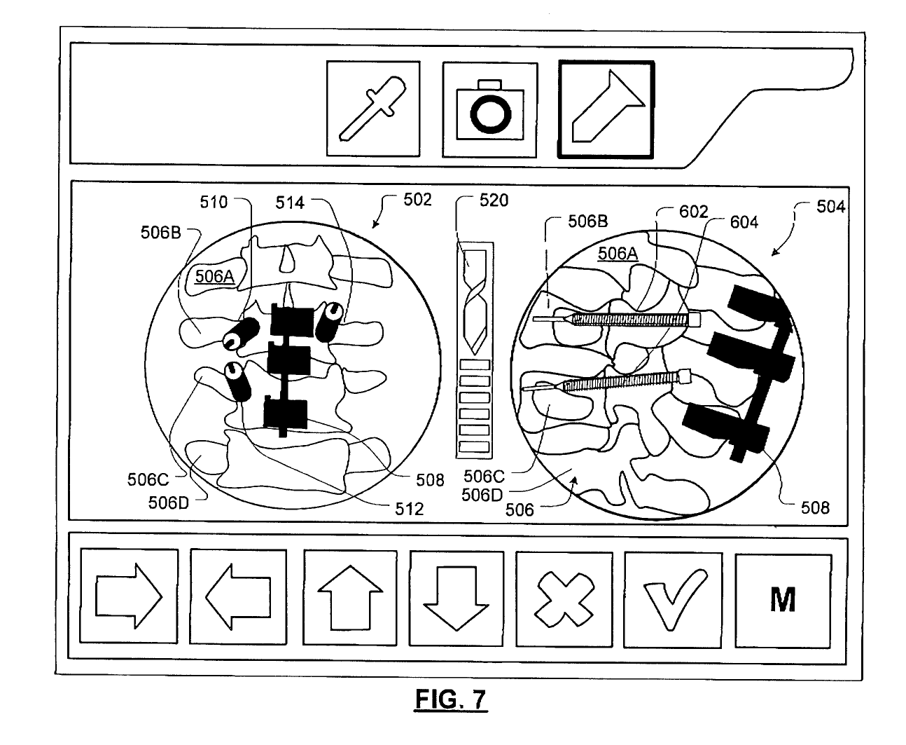

FIG. 7 shows a display screen displayed by the GUI 112 when the menu option for implant installation 254 (FIG. 3B) is selected. The other two icons are not available for selection, but are represented in a disabled state in accordance with the present embodiment. As in FIG. 6, a left side of the information pane 312 displays an AP view of the part of the patient subjected to the surgical procedure while the left side of the information pane 312 displays a lateral view. As shown in FIG. 3B, the steps involved in implant installation 254 include instrument selection and implant insertion. After the appropriate instrument has been selected for inserting the implant (a screwdriver bit on the U-Handle, for example), the surgeon aligns a screw with a first of the pedicle holes prepared as described above with reference to FIG. 6. The binocular camera 116 (FIG. 1) detects a location and orientation of the selected instrument and interprets a relative location of the instrument to generate a virtual image of the implant 602, 604 as it is being inserted into the pedicle hole. As shown in FIG. 7, a first implant 604 has been inserted. A second implant 602 is in the process of being inserted. A virtual representation of the hole drilled to receive the implant is displayed in a first color. A relative position of the implant with respect to the target position is displayed in a second color. These virtual images are automatically generated by the CAS and displayed by the GUI 112, and the surgeon is able to track implant alignment with the pedicle hole. Thus, a precision placement of the implant is assured. Subsequent to placing all implants, the medical record is documented by acquiring implant images as explained above with reference to FIG. 3B. After the images are acquired and stored, the GUI 112 displays a “Quit Application” option 212 (FIG. 3B), as explained above.

The invention therefore provides an expert system guided graphical user interface that facilitates surgical procedures by guiding a surgical team 110 through a surgical procedure, while providing critical information respecting the planning, preparation and placement of implants to ensure success of the surgery.

The embodiment(s) of the invention described above is(are) intended to be exemplary only. The scope of the invention is therefore intended to be limited solely by the scope of the appended claims.

I/We claim:

1. A graphical user interface (GUI) for guiding a surgical team in the performance of a surgical procedure, the GUI comprising:
a series of visual display screens for providing information related to respective steps required to perform the surgical procedure, and for displaying to the surgical team representations of selected surgical instruments used during the surgical procedure; and
means for permitting the surgical team to advance through the series of visual display screens as each of the respective steps is successfully completed.

2. A GUI as claimed in claim 1 wherein the series of visual display screens are organized in sequential sub-series, comprising: instrument calibration, patient imaging, implant site planning, implant site preparation, and implant installation.

3. A GUI as claimed in claim 2 further comprising menu selections and display screens for assisting the surgical team to select, and calibrate the selected surgical instruments used to perform the surgical procedure.

4. A GUI as claimed in claim 3 further comprising menu selections and display screens for assisting the surgical team in acquiring images of a part of the patient, validating the images, transforming the images, and clearing an images bank.

5. A GUI as claimed in claim 4 wherein the menu selections and display screens for transforming the images permit the surgical team to select acquired images and assist the surgical team in any one of rotating, flipping and restoring a deleted image.

6. A GUI as claimed in claim 4 wherein the display screens for assisting the surgical team in implant site planning comprise display screens that display images of the part of the patient overlaid with a planned implant location generated in response to input by the surgical team using one of the calibrated instruments.

7. A GUI as claimed in claim 4 wherein the display screens for assisting the surgical team in implant site preparation comprise display screens that display images of the part of the patient overlaid with a planned implant location, and a virtual path of an instrument used to prepare the implant site as the instrument is used by the surgical team to prepare the actual implant site.

8. A GUI as claimed in claim 7 further comprising a depth gauge for indicating a depth of the instrument used to prepare the actual implant site.

9. A GUI as claimed in claim 4 wherein the display screens for assisting the surgical team in implant installation comprise display screens that display images of the part of the patient overlaid with an image of the planned implant location, an image of the implant site prepared using one of the surgical instruments, and a virtual image of a path of the implant as the implant is installed into the prepared implant site.

10. A GUI as claimed in claim 9 further comprising menu selections and display screens for assisting the surgical team to acquire images of the inserted implant.

11. A method for guiding a surgical team in performing a surgical procedure using a graphical user interface (GUI), the method comprising steps of:
providing the surgical team with information related to respective procedure steps required to perform the surgical procedure using the GUI;
displaying to the surgical team representations of selected surgical instruments used during the surgical procedure, in alignment with a visual image of a part of a patient that is subject to the surgical procedure; and
advancing through a series of visual displays designed for the surgical procedure, as each of the respective procedure steps is successfully completed.

12. A method as claimed in claim 11 wherein the step of providing the surgical team with information comprises steps of providing visual and audio information related to instrument calibration, patient imaging, implant site planning, implant site preparation, and implant installation.

13. A method as claimed in claim 12 wherein the step of providing video and audio information related to instrument calibration comprises steps of:
prompting the surgical team to identify a selected surgical instrument to be calibrated;
prompting the surgical team to connect an instrument locator to the identified instrument, the instrument locator being adapted for identifying a position and orientation of the instrument;
prompting the surgical team to move the identified instrument with the connected locator into a field of view of a camera used to acquire images of the instrument locator; and
displaying an oriented image of a virtual representation of the position and orientation of the instrument.

14. A method as claimed in claim 13 wherein the step of providing the surgical team with visual and audio information related to patient imaging comprises steps of:
prompting and guiding the surgical team to acquire at least two images of the part of the patient; and
prompting and guiding the surgical team through a procedure for validating the respective images by placing a calibrated instrument on the part of the patient, and comparing an actual location of the calibrated instrument with a position of the virtual representation of the instrument with respect to an oriented image of a three-dimensional virtual representation of the part of the patient that is displayed by the GUI, the virtual representation generated from the at least two acquired images.

15. A method as claimed in claim 14 further comprising steps of:
providing menu options to permit the surgical team to transform images by manipulating the GUI to perform one of rotating, flipping or restoring an image; and
providing menu options to permit the surgical team to manipulate the GUI to clear an images bank after the surgical procedure is completed.

16. A method as claimed in claim 12 wherein the step of providing visual and audio information related to implant site planning comprises steps of:
displaying a menu to permit the surgical team to select an implant site planning option;
prompting the surgical team to place a calibrated instrument on the part of the patient where an implant is to be inserted;
prompting the surgical team to select a type and size of an implant to be inserted; and
displaying, in at least two views of the part of the patient, a virtual image of the selected implant in alignment with an orientation of the instrument with respect to the patient, to permit the surgical team to evaluate the planned implant site and selected implant type and size.

17. A method as claimed in claim 12 wherein the step of providing visual and audio information related to implant site preparation comprises steps of:
displaying a menu option to permit the surgical team to select an implant site preparation option;
prompting the surgical team to select a calibrated instrument for preparing a site where an implant is to be inserted; and
displaying, in at least two views of the part of the patient, a virtual image of the selected tool and its alignment with a planned location of the implant, to permit the surgical team to prepare the implant site.

18. A method as claimed in claim 17 further comprising steps of:
displaying a visual guide used to indicate to the surgical team a distance of travel of the calibrated instrument used to prepare the implant site; and
dynamically updating the visual guide to indicate to the surgical team the distance of travel of the instrument as the instrument is used to prepare the implant site.

19. A method as claimed in claim 18 wherein the calibrated instrument is one of a drill and an awl-tipped tool, and the step of displaying comprises a step of displaying a drill depth guide to indicate to the surgical team a depth of a hole drilled for the implant.

20. A method as claimed in claim 12 wherein the step of providing visual and audio information related to implant insertion comprises steps of:
displaying a menu option to permit the surgical team to select an implant insertion option;
prompting the surgical team to select a calibrated instrument used to insert the selected implant;
prompting the surgical team to insert the implant; and
displaying, in at least two views of the part of the patient, a virtual representation of the selected implant as it is inserted, by computing a virtual path of the implant by tracking a path of the instrument used to insert the implant into a prepared implant site.

21. A system for performing a computer-assisted surgical procedure, the system comprising:
a computer including a video display supporting a graphical user interface (GUI) for guiding a surgical team performing the surgical procedure wherein the GUI includes a series of visual display screens for providing information related to respective steps required to perform the surgical procedure, and for displaying to the surgical team virtual images of selected surgical instruments used during the surgical procedure in relative alignment with an oriented image of a virtual three-dimensional representation of a part of a patient subject to the surgical procedure;
means for determining a location of the selected surgical instruments with respect to the part of the patient that is subject to the surgical procedure;
means for acquiring images of the part of the patient and processing the images to generate the virtual three-dimensional representation of the part of the patient; and
means for permitting the surgical team to advance through the series of visual display screens as each of the respective steps is completed.

22. A system as claimed in claim 21 wherein the means for acquiring images of the part of the patient comprises a fluoroscope connected to the computer.

23. A system as claimed in claim 21 wherein the means for acquiring the location of the selected surgical instruments with respect to the patient comprises:
a binocular visual system connected to the computer; and
a light-reflective reference tool connected to the surgical instrument that identifies an orientation and position of the surgical instrument.

24. A system as claimed in claim 21 further comprising a data network for connecting a remote data source to the computer.

25. A system as claimed in claim 24 wherein the data network is the Internet.

26. A system as claimed in claim 21 wherein the means for permitting the surgical team to advance through the series of visual display screens comprises a manual input device connected to the computer.

27. A system as claimed in claim 21 wherein the means for permitting the surgical team to advance through the series of visual display screens comprises a foot-operated input device connected to the computer.
